(19) United States
(12) Patent Application Publication
(10) Pub. No.: US 2024/0100299 A1
(43) Pub. Date: Mar. 28, 2024

(54) ASPIRATION CATHETER WITH SHAPED TIP AND ANGLED CUT

(71) Applicant: Acotec Technologies Limited, San Jose, CA (US)

(72) Inventor: Jon Schabert, San Jose, CA (US)

(73) Assignee: Acotec Technologies Limited, San Jose, CA (US)

(21) Appl. No.: 18/470,123

(22) Filed: Sep. 19, 2023

Related U.S. Application Data
(60) Provisional application No. 63/410,622, filed on Sep. 28, 2022.

(51) Int. Cl. A61M 25/00

(52) U.S. Cl. CPC A61M 25/0068; A61M 25/0074

(57) ABSTRACT

A catheter includes an elongate tubular body having a side wall defining a lumen extending between a proximal region and a distal region. The distal region comprises a tip portion capable of deflecting away from the proximal region when the tip portion is unconstrained, thereby forming a curved shape of the tip portion. The side wall of the elongate tubular body axially terminates at the tip portion with a slant cut, thereby forming an end surface of the tip portion defining an opening in a non-circular shape.

### TECHNICAL FIELD

This application relates generally to medical devices and methods of making and using medical devices. In particular, various embodiments of a catheter device and a method of removing obstructions such as clots from human blood vessels are described.

### BACKGROUND

Thrombi or blood clots can cause various medical disorders including peripheral thrombosis, pulmonary embolism, strokes, heart attack, and the like. Blood clots in large veins of the legs and pelvis may give rise to deep venous thrombosis (DVT), which causes harm by obstructing drainage of venous blood from the legs, leading to swelling, ulcers, pain, and infection. DVT may also serve as a source of occlusive materials to travel to other parts of the body including the lungs, heart, brain, and other organs. In pulmonary embolism (PE), occlusive materials obstruct the main or large branch pulmonary arteries, compromising total blood flow within the lungs and therefore the entire body, resulting in low blood pressure and shock. If occlusive materials are in large to medium pulmonary artery branches, they can prevent a significant portion of the lung from participating in exchange of gases to the blood, resulting in low blood oxygen and buildup of blood carbon dioxide. Occlusive materials in neuro vasculature can block blood to the brain, giving rise to stroke causing brain damage, long-term disability, or death.

Various medical procedures are known and have been used in treating deep venous thrombosis (DVT), pulmonary embolism (PE), strokes, and the like. For example, in a thrombectomy procedure for treating deep venous thrombosis (DVT), an aspiration catheter may be used to remove clots from vessels. While significant advancement has been achieved, there remains a general need for improvement. For example, it would be desirable to provide an aspiration catheter which can reach restricted spaces in smaller vessels and allow for increased aspiration force and torquing response.

### SUMMARY

In one aspect, embodiments of the disclosure feature a catheter. In general, an embodiment of the catheter comprises an elongate tubular body having a side wall defining a lumen extending between a proximal region and a distal region. The distal region comprises a tip portion capable of deflecting away from the proximal region when the tip portion is unconstrained, thereby forming a curved shape of the tip portion. The side wall of the elongate tubular body axially terminates at the tip portion with a slant cut, thereby forming an end surface of the tip portion defining an opening in a non-circular shape.

In various embodiments of the aspect, the tip portion is configured to deflect away from a longitudinal axis of the proximal region at an angle ranging from 5 degrees to 315 degrees.

In various embodiments of the aspect, the inclined end surface of the tip portion forms an angle ranging from 20 degrees to 80 degrees relative to an inner or outer surface of the side wall of the tubular body.

In various embodiments of the aspect, the catheter further comprises at least one stretch resistant filament extending from the proximal region to the distal region of the elongate tubular body.

In various embodiments of the aspect, the at least one stretch resistant filament may be arranged such that a distal portion of the at least one stretch resistant filament extends along inside the curved shape of the tip portion.

In various embodiments of the aspect, the at least one stretch resistant filament can be configured to provide a tensile strength ranging from 5 pounds to 25 pounds.

In various embodiments of the aspect, the lumen of the catheter has a diameter ranging from 0.164 inches to 0.295 inches.

In a further aspect, embodiments of the disclosure feature a catheter device. The catheter device comprises a first catheter having a lumen, and a second catheter configured to be positioned within the lumen of the first catheter and longitudinal movable relative to the first catheter. The second catheter comprises an elongate tubular body having a side wall defining a lumen extending between a proximal region and a distal region of the elongate tubular body. The distal region of the tubular body comprises a tip portion capable of deflecting away from the proximal region of the tubular body when the tip portion is unconstrained by the first catheter, thereby forming a curved shape of the tip portion. The side wall of the elongate tubular body axially terminates at the tip portion with a slant cut, thereby forming an end surface of the tip portion defining an opening in a non-circular shape.

In various embodiments of the aspect, the side wall of the first catheter axially terminates with a straight cut, forming an end surface of the first catheter defining an opening in a generally circular shape.

In various embodiments of the aspect, the lumen of the first catheter has a diameter ranging from 0.190 inches to 0.320 inches, and the lumen of the second catheter has a diameter ranging from 0.164 inches to 0.295 inches.

In various embodiments of the aspect, the tip portion of the second catheter is configured to deflect away from the proximal region of the second catheter at an angle ranging from 5 degrees to 315 degrees.

In various embodiments of the aspect, the end surface of the tip portion of the second catheter forms an angle ranging from 20 degrees to 80 degrees relative to an inner or outer surface of the side wall of the second catheter.

In various embodiments of the aspect, the catheter device further comprises at least one stretch resistant filament extending from the proximal region to the distal region of the second catheter.

In various embodiments of the aspect, the at least one stretch resistant filament is arranged such that a distal portion of the at least one stretch resistance filament extends along inside the curved shape of the tip portion of the second catheter.

In various embodiments of the aspect, the at least one stretch resistant filament is configured to provide a tensile strength ranging from 5 pounds to 25 pounds.

This Summary is provided to introduce selected aspects and embodiments of this disclosure in a simplified form and is not intended to identify key features or essential characteristics of the claimed subject matter, nor is it intended to be used as an aid in determining the scope of the claimed subject matter. The selected aspects and embodiments are presented merely to provide the reader with a brief summary of certain forms the invention might take and are not intended to limit the scope of the invention. Other aspects and embodiments of the disclosure are described in the section of Detailed Description.

These and various other aspects, embodiments, features, and advantages of the disclosure will become better understood upon reading of the following detailed description in conjunction with the accompanying drawings.

### BRIEF DESCRIPTION OF THE DRAWINGS

FIG. 1 illustrates an example aspiration system according to embodiments of the disclosure.

FIGS. 2A-2B illustrate an example catheter device according to embodiments of the disclosure.

FIG. 3 illustrates an example aspiration catheter according to embodiments of the disclosure.

FIG. 4 illustrate an enlarged cross-section view of a portion of an example aspiration catheter according to embodiments of the disclosure.

FIG. 5A illustrates an opening end of an example aspiration catheter with an angled cut of the side wall at the tip of the catheter. FIG. 5B illustrates an opening end of an example aspiration catheter with a straight cut of the side wall at the tip of the catheter.

### DETAILED DESCRIPTION OF EMBODIMENTS

With reference to the figures, various embodiments of an aspiration catheter and device will now be described. The figures are intended to facilitate description of embodiments of the disclosure and are not necessarily drawn to scale. Certain specific details may be set forth in the figures and description to provide a thorough understanding of the disclosure. It will be apparent to one of ordinary skill in the art that some of these specific details may not be employed to practice embodiments of the disclosure. In other instances, structures, materials, components, systems, and/or operations often associated with medical procedures may not be shown or described in detail to avoid unnecessarily obscuring description of embodiments of the disclosure.

Embodiments of the disclosure provide an aspiration catheter having a shaped tip with an angled cut. The aspiration catheter comprises a distal tip portion capable of deflecting away the longitudinal axis of the catheter when the distal tip portion is unconstrained, forming a curved shape of the tip portion. The side wall of the catheter is angled or slant cut at the distal tip, forming an inclined end surface defining an opening in a non-circular shape. The aspiration catheter of the disclosure allows for increased ability to sweep around a blood vessel to aspirate clots, with the facing direction of the angled cut being controlled by the pre-shaped curve. One or more stretch resistant fibers or filaments can be included in the aspiration catheter, e.g., extending along the inside curve of the shaped tip. The stretch resistant filament(s) can ensure the orientation of the angle cut to be maintained or controlled when the aspiration catheter is torqued to direct the distal tip towards the desired area to aspirate clots. The stretch resistant filament(s) can also increase the response between torquing of the proximal portion of the catheter and movement or rotation of the distal tip. The aspiration catheter of the disclosure provides for greater suction force to better remove clots. The pre-shaped distal tip with an angled cut allows for aspiration force to reach areas with a shorter length of the catheter, which can be extremely beneficial in performing procedures in smaller vessels or restricted spaces such as neuro and pulmonary vasculatures.

While the term “aspiration” is used in describing various embodiments of disclosure, the catheter, system, and method disclosed herein can be used in other medical procedures other than or in addition to aspiration. For example, the catheter can be used to deliver therapeutical agents, fluids or medical devices to a treatment site in a vasculature, or as a support catheter to provide a conduit that facilitates and guides delivery of other devices such as guidewires and interventional devices. Further, while embodiments of the disclosure may be described in conjunction with an aspiration thrombectomy procedure in a particular anatomy, the catheter, system, and method described herein can be configured to access, deliver agents or devices, or perform procedures in various anatomies including e.g., peripheral, pulmonary, coronary, and neurovascular anatomies.

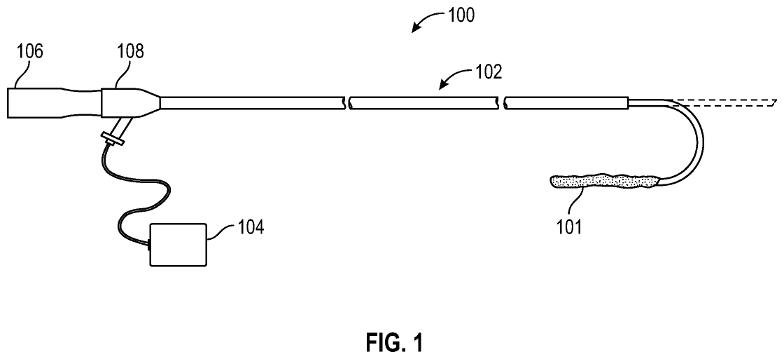

FIG. 1 depicts an example aspiration system 100 according to embodiments of the disclosure. The aspiration system 100 can be used to remove occlusions such as a clot 101 in a deep vein, pulmonary artery, neuro vasculature, or other treatment sites. As shown, the aspiration system 100 in general comprises a catheter device 102, a vacuum source 104, a torquing device 106, and a hub 108 operably connecting the catheter device 102, the vacuum source 104, the torquing device 106, and other devices via suitable tubing and/or connectors. The vacuum source 104 may be provided by a vacuum pump, a syringe, or other suitable vacuum sources. The torquing device 106 may include a rotation mechanism operable to torque the catheter device 102 to facilitate navigation of the device in a patient's anatomy and aspiration of clots. Therefore, in operation, the catheter device 102 may access to a treatment site and operate to aspirate and remove an occlusion 101 upon application of a negative pressure provided by the vacuum source 104. While not shown in FIG. 1, the aspiration system 100 may also include a fluid source operably connected to the hub 108 via a connection port. The fluid source may provide saline, blood, or other fluids which may be needed for performing the procedure.

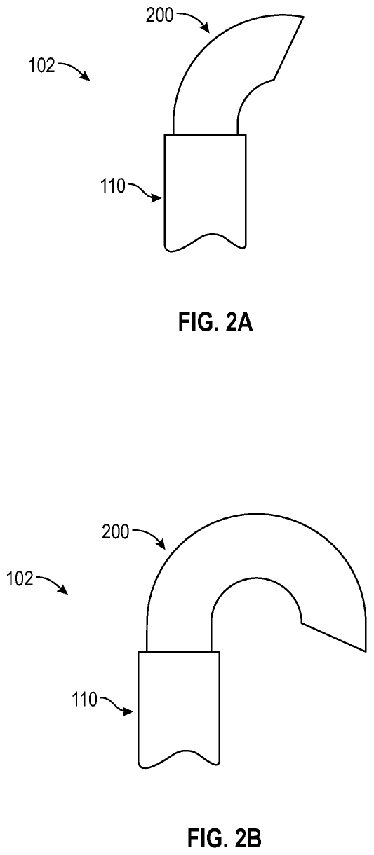

With reference to FIGS. 1 and 2A-2B, the catheter device 102 may include a larger first catheter 110 and a smaller second catheter 200. The smaller second catheter 200 is configured to be positioned within the lumen of the larger first catheter 110 and longitudinally movable relative to the first catheter 110. The proximal end of the first and/or the second catheters 110, 200 may be coupled to the hub 108 e.g., via a valve, forming a fluidic communication between the the vacuum source 104 and the first and/or second catheters 110, 200. The diameter and the length of the first and second catheters 110, 200 may be chosen or determined at least partially based on the treatment site that the catheter device 102 is designed to reach. By way of example, for treatment of deep veinous thrombosis (DVT), the lumen of the first catheter 110 may have a diameter ranging from 0.190 inches to 0.320 inches, and the lumen of the second catheter 200 may have a diameter ranging from 0.164 inches to 0.295 inches.

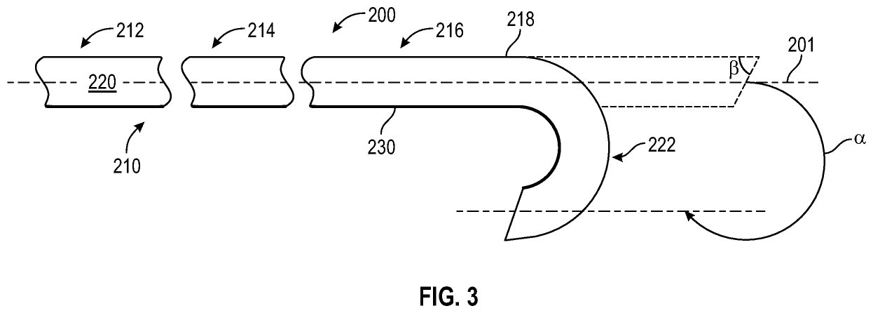

With reference to FIG. 3, the second catheter 200 comprises an elongate tubular body 210 having a proximal region 212, an intermediate region 214, and a distal region 216. The elongate tubular body 210 comprises a side wall 218 defining a lumen 220 extending between the proximal region 212 and the distal region 216. According to embodiments of the disclosure, the distal region 216 of the tubular body comprises a tip portion 222 capable of deflecting away from the proximal region 212 or intermediate region 214 when the tip portion 222 is unconstrained e.g., when exiting the lumen of the larger first catheter 110 as shown in FIGS. 2A-2B. When constrained e.g., in a larger catheter, the tip portion 222 at the distal region 216 can be generally aligned with the proximal and the intermediate regions 212, 214 or the longitudinal axis 201 of the proximal and intermediate regions 212, 214, as indicated by the phantom lines in FIG. 3. When unconstrained, the tip portion 222 of the distal region 216 deflects away from the longitudinal axis 201 of the proximal and intermediate regions 212, 214, forming a curved shape as shown in FIG. 3.

According to embodiments of the disclosure, the tip portion 222 at the distal region 216 can deflect away from the longitudinal axis 201 of the proximal or intermediate region 212, 214 at a deflection angle (α) ranging from 5 degrees to 315 degrees. In some embodiments, the deflection angle (α) may be greater than 315 degrees and/or smaller than 5 degrees. In some embodiments, the deflection angle (α) may range from 45 degrees to 315 degrees. In some specific embodiments, the tip portion 222 may deflect away from the longitudinal axis 201 of the proximal or intermediate region at a deflection angle (α) of 5 degrees, 15 degrees, 25 degrees, 35 degrees, 45 degrees, 135 degrees, 225 degrees, 315 degrees, or any degrees therebetween. The curved shape of the tip portion 222 can be in various shapes, including generally O-shape, U-shape, L-shape, partially O-shape, partially U-shape, partially L-shape, or any combination of the O-. U-, and L-shapes, as known to one of ordinary skill in the art. For purpose of illustration, a curved shape in a partially O-shape is shown in FIG. 3. It should be noted that the principle and the appended claims of the disclosure are not limited to the specific curved shape of the tip portion.

With reference to FIG. 3, according to embodiments of the disclosure, the side wall 218 of the elongate tubular body of the second catheter may be angled cut or slant cut at the distal tip portion 222, or the side wall 218 may axially terminate with an inclined end surface, forming a distal tip portion 222 with an opening having a non-circular shape e.g., an oval. By way of example, the cut angle (β) may range from 20 degrees to 80 degrees with respect to the outer or inner surface of the side wall of the catheter. In some embodiments, the cut angle (β) may be greater than 80 degrees and/or smaller than 20 degrees. In some specific embodiments, the cut angle (β) may be 20 degrees, 25 degrees, 30 degrees, 35 degrees, 40 degrees, 45 degrees, 50 degrees, 65 degrees, 70 degrees, 75 degrees, 80 degrees, or any degrees therebetween.

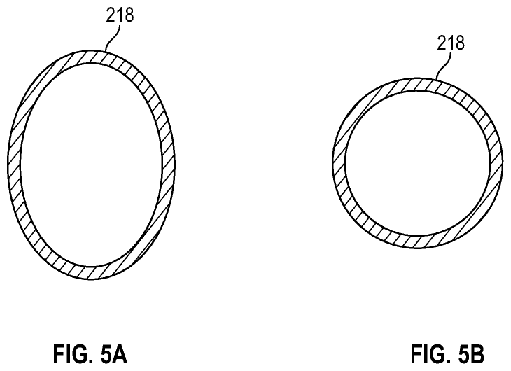

An angled cut or slant cut of the side wall 218 of the aspiration catheter 200 forms a distal tip portion 222 with an opening having a non-circular shape e.g., an oval. In comparison, a flat or straight cut of the side wall of a catheter would provide a distal tip with an opening having a circular shape. The term “flat cut” or “straight cut” is used herein to refer to a cut of a side wall at an angle of 90 degrees with respect to the outer or inner surface of the side wall. The term “angled cut” or “slant cut” is used herein to refer to a cut of a side wall at a non-normal degree e.g., a degree from 25 degrees to 75 degrees with respect to the outer or inner surface of the side wall. FIG. 5A illustrates an end view of a distal tip with an angled cut of the side wall. FIG. 5B illustrates an end view of a distal tip with a straight cut of the side wall. As shown, a tip portion with an angled cut defines an opening with a non-circular shape (FIG. 5A) whereas a tip portion with a flat or straight cut defines an opening with a circular shape (FIG. 5B). The opening area of a catheter tip with an angled cut is greater than the opening area of a catheter tip with a straight cut. As such, the aspiration catheter having a distal tip with an angled cut allows for greater suction force to better remove clots from the blood vessel.

An aspiration catheter having a shaped distal tip with an angled cut can be used with a catheter having a distal tip with a straight cut. Therefore, in embodiments, a catheter device 102 of the disclosure comprises a first catheter 110 and a second catheter 200 positioned within the lumen of the first catheter 110, as shown FIGS. 2A-2B. The side wall of the first catheter 110 may be straight cut at the distal end, or axially terminates with a straight end surface forming a distal tip portion with an opening in a circular shape. The side wall of the second catheter 200 may be angled cut or slant cut at the distal end, or axially terminates with an inclined end surface forming a distal tip portion with an opening in a non-circular shape. In addition, the distal tip 222 of the second catheter 200 with an angled cut may be configured to have a curved shape when unconstrained by the first catheter 110. The catheter device 100 of the disclosure allows for aspiration force to reach areas with a less length of the second catheter 200 existing out of the first catheter 110, which can be extremely useful in conducting aspiration in smaller vessels or restricted spaces such as in the neuro vasculatures and the lungs.

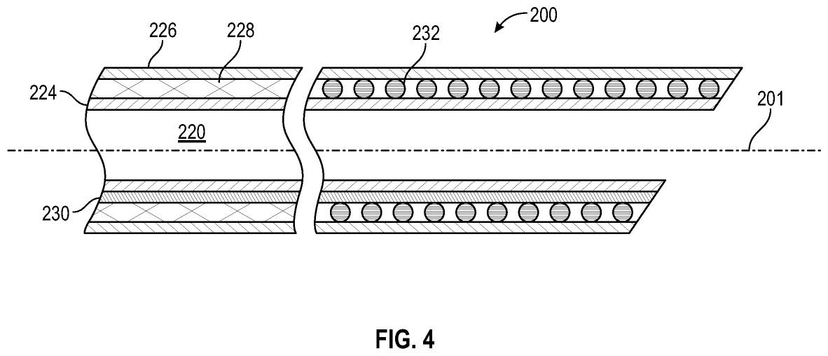

With reference to FIG. 4, an example aspiration catheter 200 of the disclosure may include one or more stretch resistant filaments 230. The one or more stretch resistant filaments 230 increase torque response of the catheter 200 and ensure that the facing direction of the distal tip with an angled cut be maintained or controlled when the catheter is torqued. As shown in FIG. 4, the aspiration catheter 200 may comprise an inner layer or liner 224, a jacket or sheath layer 226, a reinforcement layer 228, and one or more stretch resistant filaments 230. At the distal region, the aspiration catheter 200 may include a coil of a memory material 232.

With reference to FIG. 4, the inner layer or liner 224 may extend the length of the aspiration catheter 200, from the proximal region 212 to the distal region 216 defining a lumen of the catheter. The inner liner 224 can be constructed from a lubricious or low-friction material to provide a smooth surface for advancement of devices or objects through the inner lumen. Suitable lubricious materials include but are not limited to polytetrafluoroethylene (PTFE), fluorinated ethylene propylene (FEP), and other suitable polymeric materials.

The jacket or sheath layer 226 can provide mechanical integrity to the aspiration catheter 200. The jacket layer 226 may be constructed from a material such as a thermoplastic elastomer (TPE) e.g., polyether block amide, thermoplastic polyurethane, polyethylene, nylon, or the like.

The reinforcement layer 228 may be incorporated between the inner liner 224 and the jacket layer 226. The reinforcement layer 228 can be constructed to prevent kinking or flattening of the inner lumen 224 of the catheter in navigation through a tortuous vasculature. The reinforcement layer 228 can be made from metal such as stainless steel, nitinol, or from polymers, or any combinations thereof. The reinforcement layer 228 can be in a structure of a braid, or flexible tubing with a laser-cut pattern, or a hypotube such as a cut hypotube or cut rigid polymer, or the like. According to embodiments of the disclosure, the reinforcement layer 228 may extend partially along the length of the catheter 200. For example, the reinforcement layer 228 may be omitted in a portion of the distal and/or proximal region of the catheter 200. As such, a coil of a memory material 232 can be disposed in the distal region of the catheter adjacent or next to the reinforcement braid 228. According to embodiments of the disclosure, a coil of a memory material 232 can be heat-set to provide a pre-determined curved shape when unconstrained. When constrained, the coil of a memory material 232 may generally align with the braid reinforcement layer 228 or the longitudinal axis of the catheter 200, as shown in FIG. 4. When unconstrained, the coil of a memory material 232 assumes the curved shape that is pre-set, as discussed above in conjunction with FIGS. 1-3.

With reference to FIG. 4, the one or more stretch resistant filaments 230 may be incorporated between the inner liner 224 and the reinforcement braid 228 and the coil of a memory material 232. The one or more stretch resistant filaments 230 may extend from the proximal region 212 to the distal region 216 of the aspiration catheter 200. According to embodiments of the disclosure, at least one stretch resistant filament 230 is arranged such that it extends along inside the curve of the shaped distal tip portion 222. As such, the stretch resistant filament 230 can lock the distal tip orientation when the catheter 200 is torqued, increasing the torquing response of the catheter 200. The one or more stretch resistant filament(s) 230 can ensure that the facing direction of the distal tip 222 with an angled cut to be maintained or controlled when the catheter 200 is torqued in directing the distal tip 222 towards the desired area to aspirate clots. Because the stretch resistant filament(s) 230 have the property or tendency to maintain themselves oriented e.g., along the inside curve of the distal tip 222, the orientation can be maintained throughout torquing of the catheter 200. The stretch resistant filament(s) 230 can also increase the response between torquing of the proximal region 212 of the catheter 200 and movement or rotation of the distal tip portion 222 because any delay in response would otherwise require “stretching” or lengthening of the stretch resistant filament(s) 230. The stretch resistant filament(s) 230 is (are) constructed to resist stretching unless being ruptured. As such, the stretch resistant filament(s) 230 can provide substantially 1:1 torque response to keep the distal tip portion 222 and the proximal/intermediate portion 212, 214 in synch during torquing of the aspiration catheter 200.

The stretch resistant filaments 230 can be constructed from metals, metal alloys, or polymers which provide stretch resistance. Suitable materials for constructing stretch resistant filaments 230 include but are not limited to polyesters, aramid fibers, or the like, which are commercially available. According to embodiments of the disclosure, the stretch resistant filaments 230 can provide a tensile strength ranging from 5 pounds 25 pounds. In some embodiments, the stretch resistant filaments 230 provide a tensile strength greater than 25 pounds or smaller than 5 pounds.

While not shown in the drawings, the aspiration catheter 200 may include one or more radiopaque markers along the length of the catheter. For instance, one or more radiopaque markers may be included at the distal region and/or the intermediate region to allow for visualization of the shaped distal tip portion respect to the intermediate region of the catheter when in use.

Various embodiments of an aspiration catheter have been described with reference to figures. It should be noted that an aspect described in conjunction with a particular embodiment is not necessarily limited to that embodiment and can be practiced in any other embodiments. The figures are intended for illustration of embodiments but not for exhaustive description or limitation on the scope of the disclosure. Alternative structures, components, and materials will be readily recognized as being viable without departing from the principle of the claimed invention.

All technical and scientific terms used herein have the meaning as commonly understood by one of ordinary skill in the art unless specifically defined otherwise. As used in the description and appended claims, the singular forms of “a,” “an,” and “the” include plural references unless the context clearly dictates otherwise. The term “or” refers to a nonexclusive “or” unless the context clearly dictates otherwise. The term “proximal” and its grammatically equivalent refers to a position, direction or orientation towards the user or physician's side. The term “distal” and its grammatically equivalent refers to a position, direction, or orientation away from the user or physician's side. The term “first” or “second” etc. may be used to distinguish one element from another in describing various similar elements. It should be noted the terms “first” and “second” as used herein include references to two or more than two. Further, the use of the term “first” or “second” should not be construed as in any particular order unless the context clearly dictates otherwise. The order in which the method steps are performed may be changed in alternative embodiments. One or more method steps may be skipped altogether, and one or more optional steps may be included. All numeric values are provided for illustration and assumed to be modified by the term “about,” whether explicitly indicated or not. The term “about” generally refers to a range of numbers that one of skill in the art would consider equivalent to the recited value e.g., having the same function or result. The term “about” may include numbers that are rounded to the nearest significant figure. The term “about” may be used to indicate a value that can include a variation of ±10% of the value modified by the term. The recitation of a numerical range by endpoints includes all numbers within that range.

Those skilled in the art will appreciate that various other modifications may be made. All these or other variations and modifications are contemplated by the inventors and within the scope of the invention.

What is claimed is:

1. A catheter comprising an elongate tubular body having a side wall defining a lumen extending between a proximal region and a distal region,
wherein the distal region comprises a tip portion capable of deflecting away from the proximal region when the tip portion is unconstrained, thereby forming a curved shape of the tip portion; and
wherein the side wall of the elongate tubular body axially terminates at the tip portion with a slant cut, thereby forming an end surface of the tip portion defining an opening in a non-circular shape.

2. The catheter of claim 1, wherein the tip portion is configured to deflect away from a longitudinal axis of the proximal region at an angle ranging from 5 degrees to 315 degrees.

3. The catheter of claim 2, wherein the inclined end surface of the tip portion forms an angle ranging from 20 degrees to 80 degrees relative to an inner or outer surface of the side wall of the tubular body.

4. The catheter of claim 3, further comprising at least one stretch resistant filament extending from the proximal region to the distal region of the elongate tubular body.

5. The catheter of claim 4, wherein the at least one stretch resistant filament is arranged such that a distal portion of the at least one stretch resistant filament extends along inside the curved shape of the tip portion.

6. The catheter of claim 5, wherein the at least one stretch resistant filament is configured to provide a tensile strength ranging from 5 pounds to 25 pounds.

7. The catheter of claim 1, further comprising at least one stretch resistant filament extending from the proximal region to the distal region of the elongate tubular body.

8. The catheter of claim 7, wherein the at least one stretch resistant filament is arranged such that a distal portion of the at least one stretch resistance filament extends along inside the curved shape of the tip portion.

9. The catheter of claim 7, wherein the lumen of the catheter has a diameter ranging from 0.164 inches to 0.295 inches.

10. The catheter of claim 1, wherein the lumen of the catheter has a diameter ranging from 0.164 inches to 0.295 inches.

11. A catheter device, comprising:
a first catheter having a lumen; and
a second catheter configured to be positioned within the lumen of the first catheter and longitudinal movable relative to the first catheter, wherein
the second catheter comprises an elongate tubular body having a side wall defining a lumen extending between a proximal region and a distal region of the elongate tubular body,
wherein the distal region of the tubular body comprises a tip portion capable of deflecting away from the proximal region of the tubular body when the tip portion is unconstrained by the first catheter, thereby forming a curved shape of the tip portion; and
wherein the side wall of the elongate tubular body axially terminates at the tip portion with a slant cut, thereby forming an end surface of the tip portion defining an opening in a non-circular shape.

12. The catheter device of claim 11, wherein the first catheter comprises a side wall defining the lumen of the first catheter, the side wall of the first catheter axially terminating with a straight cut, forming an end surface of the first catheter defining an opening in a generally circular shape.

13. The catheter device of claim 11, wherein the lumen of the first catheter has a diameter ranging from 0.190 inches to 0.320 inches, and the lumen of the second catheter has a diameter ranging from 0.164 inches to 0.295 inches.

14. The catheter device of claim 11, wherein the tip portion of the second catheter is configured to deflect away from the proximal region of the second catheter at an angle ranging from 5 degrees to 315 degrees.

15. The catheter device of claim 14, wherein the end surface of the tip portion of the second catheter forms an angle ranging from 20 degrees to 80 degrees relative to an inner or outer surface of the side wall of the second catheter.

16. The catheter device of claim 15, further comprising at least one stretch resistant filament extending from the proximal region to the distal region of the second catheter.

17. The catheter device of claim 16, wherein the at least one stretch resistant filament is arranged such that a distal portion of the at least one stretch resistance filament extends along inside the curved shape of the tip portion of the second catheter.

18. The catheter device of claim 17, wherein the at least one stretch resistant filament is configured to provide a tensile strength ranging from 5 pounds to 25 pounds.

19. The catheter device of claim 11, further comprising at least one stretch resistant filament extending from the proximal region to the distal region of the elongate tubular body of the second catheter.

20. The catheter device of claim 19, wherein the at least one stretch resistant filament is arranged such that a distal portion of the at least one stretch resistance filament extends along inside the curved shape of the tip portion of the second catheter.
